(19) United States
(12) Patent Application Publication
(10) Pub. No.: US 2023/0212495 A1
(43) Pub. Date: Jul. 6, 2023

(54) SEMI-AUTOMATED HOLLOW FIBER SYSTEM FOR VIRAL TRANSDUCTION

(71) Applicant: Takeda Pharmaceutical Company Limited, Osaka (JP)

(72) Inventors: Nathan Moore, Lexington, MA (US); Fabio Fachin, Lexington, MA (US)

(21) Appl. No.: 18/000,725

(22) PCT Filed: Jun. 10, 2021
(86) PCT No.: PCT/JP2021/022031

§ 371 Date: Dec. 5, 2022

Related U.S. Application Data
(60) Provisional application No. 63/037,377, filed on Jun. 10, 2020.

(51) Int. Cl. C12M 1/12; C12M 1/00

(52) U.S. Cl. CPC C12M 25/10; C12M 29/00

(57) ABSTRACT

A system for introducing a vector into includes a filter module defining an intra-capillary space and an extra-capillary space separated from the intra-capillary space by a porous membrane. The system also includes a pair of intra-capillary ports fluidly coupled to opposite ends of the intra-capillary space and each receiving a transduction media, cells, and a vector. The system also includes a pair of extra-capillary ports coupled to opposite ends of the extra-capillary space and in fluid-communication with a source of extra-capillary media and a waste container.

### CROSS REFERENCE TO RELATED APPLICATIONS

This application claims priority under 35 U.S.C. § 119(e) to U.S. Provisional Application 63/037,377, filed on Jun. 10, 2020. The disclosure of this prior application is considered part of the disclosure of this application and is hereby incorporated by reference in its entirety.

### TECHNICAL FIELD

The present disclosure relates to a semi-automated method and system for viral transduction using a hollow fiber filter module.

### BACKGROUND

Cell therapies take advantage of the natural transduction process, using virus particles modified for safety and functionality as a delivery vehicle (vector) for introducing therapeutic genes into a patient's cells. Viral vector transduction is currently the most frequently used method in cell therapy manufacturing for introducing therapeutic genetic material.

Current manufacturing transduction processes are labor intensive and inefficient in the use of viral vectors, contributing to the high cost of manufacturing cell therapies and extending the time required to produce these therapies. Accordingly, there are significant limitations in the current state of the art in manufacturing transduction processes.

### SUMMARY

One aspect of the disclosure provides a system for introducing a vector into cells. The system includes a filter module defining an intra-capillary space and an extra-capillary space separated from the intra-capillary space by a porous membrane. The system also includes a pair of intra-capillary ports fluidly coupled to opposite ends of the intra-capillary space and each receiving a transduction media, cells, and a vector. The system also includes a pair of extra-capillary ports coupled to opposite ends of the extra-capillary space and in fluid-communication with a source of extra-capillary media and a waste container.

This aspect of the disclosure may include one or more of the following optional features. In some examples, the system includes a harvest container in fluid communication with at least one of the intra-capillary ports. In some implementations, the system includes an intra-capillary pump operable to provide a flow of each of the transduction media, the cells, and the vector to at least one of the intra-capillary ports. Optionally, the intra-capillary pump is operable in a first state to provide the cells and the vector to the intra-capillary ports during a first period of time and in a second state to provide the transduction media to the intra-capillary ports during a second period of time.

In some examples, the system includes waste container in communication with the extra-capillary space through at least one of the extra-capillary ports. In some implementations, the system includes an extra-capillary pump operable to provide a flow of the extra-capillary media to each of the extra-capillary ports. In some configurations, includes system an extra-capillary pump operable to provide a flow of a waste fluid from the extra-capillary ports to the waste container.

In some implementations, the porous membrane is cylindrical. In some examples, the porous membrane comprises pores that allow particles with a size of less than about 50 kDa to pass through the pores from the intra-capillary space. In some configurations, the intra-capillary space defines a transduction zone.

Another aspect of the disclosure provides a system for introducing a viral or a non-viral vector into cells. The system includes a hollow fiber defining an intra-capillary space extending from a first end to a second end. The system also includes a casing enclosing the one or more hollow fibers to define an extra-capillary space between the hollow fiber and the casing from the first end to the second end, the casing comprising a first port in fluid communication with the intra-capillary space adjacent to the first end and a second port in fluid communication with the intra-capillary space adjacent to the second end. The system also includes a transduction media source in fluid communication with the intra-capillary space through each of the first port and the second port. The system further includes a cell source including cells and in fluid communication with the intra-capillary space through each of the first port and the second port. The system also includes a virus source including a viral or non-viral vector and in fluid communication with the intra-capillary space through each of the first port and the second port.

This aspect of the disclosure may include one or more of the following optional features. In some examples, the system includes a harvest container in fluid communication with the intra-capillary space through at least one of the first port and the second port. In some implementations, the system comprising an intra-capillary pump including an inlet in fluid communication with each of the transduction media source, the cell source, and the virus source. In some examples, the intra-capillary pump includes a first outlet in fluid communication with the intra-capillary space through the first port and a second outlet in fluid communication with the intra-capillary space through the second port.

In some configurations, the casing includes a third port in communication with the extra-capillary space and the system further includes a waste container in communication with the extra-capillary space through the third port. In some examples, the system includes an extra-capillary media source in fluid communication with the extra-capillary space through the third port. In some configurations, the third port is disposed adjacent to the first end of the intra-capillary space and the system further comprises a fourth port in fluid communication with the extra-capillary space and disposed adjacent to the second end of the intra-capillary space. In some examples, each of the waste container and the extra-capillary media source are in communication with the extra-capillary space through each of the third port and the fourth port.

In some configurations, the hollow fiber includes a plurality of hollow fibers. In some implementations, the hollow fiber includes pores that allow particles with a size of less than about 50 kDa to pass through the pores from the intra-capillary space.

Yet another aspect of the disclosure provides a method of introducing a viral or non-viral vector into cells using a hollow fiber defining an intra-capillary space extending from a first end to a second end and an extra-capillary space surrounding the intra-capillary space from the first end to the second end. The method includes loading a viral or non-viral vector into the intra-capillary space of the hollow fiber and loading cells into the intra-capillary space of the hollow fiber.

This aspect of the disclosure may include one or more of the following optional features. In some examples, loading the viral or non-viral vector into the intra-capillary space includes loading the viral or non-viral vector from at least one of the first end and the second end of the intra-capillary space. In some implementations, loading the viral or non-viral vector into the intra-capillary space includes loading the viral or non-viral vector from each of the first end and the second end of the intra-capillary space. In some configurations, loading the cells into the intra-capillary space includes loading the cells from at least one of the first end and the second end of the intra-capillary space.

In some examples, loading the cells into the intra-capillary space includes loading the cells from each of the first end and the second end of the intra-capillary space. Optionally, the method may further include transducing the cells within the intra-capillary space of the hollow fiber and harvesting the transduced cells from the intra-capillary space of the hollow fiber. In some examples, harvesting the transduced cells from the intra-capillary space includes loading a flushing fluid into the extra-capillary space of the hollow fiber. In some implementations, harvesting the transduced cells from the intra-capillary space includes loading a flushing fluid into the intra-capillary space from one of the first end or the second end.

In some examples, the method includes collecting a waste from the extra-capillary space. In some implementations, the cells and viral or non-viral vector are loaded simultaneously. In some configurations, the cells and viral or non-viral vector are loaded separately. In some implementations, the cells are loaded prior to the viral or non-viral vector. In some configurations, the viral or non-viral vector are loaded prior to the cells.

In some examples, the cells are loaded at a concentration ranging between 1×103 to 1×1010 cells/ml. In some implementations, loading the cells includes loading cells at a rate that is a function of a size of inner surface area of the hollow fiber. In some configurations, the viral or non-viral vector are loaded as viral particles. In some examples, the viral or non-viral vector are loaded as nucleic acid vector.

In some examples, the method includes loading the cells and the viral or non-viral vector at a flow rate per square centimeter of an inner surface area of the hollow fiber of between about 5-100 μl/min/cm2. In some examples, loading at a flow rate per square centimeter of an inner surface area of the hollow fiber is between about 5-20 μl/min/cm2. In some implementations, the vector is derived from a lentivirus, a retrovirus, an adenovirus, an adeno-associated virus, or a hybrid virus. In some examples, the vector is a retrovirus. In some implementations, the vector is a lentivirus. In some examples, the vector comprises nanoparticles, liposomes, lipid particles, carbon, non-reactive metals, gelatin and/or polyamine nanospheres.

In some implementations, cells and viral vector are loaded into the intra-capillary space at a multiplicity of infection (MOI) ranging from about 0.25 to about 4.0. In some examples, the cells and viral vector are loaded into the intra-capillary space at an MOI of about 2.5. In some configurations, the cells are B-cells, T cells, NK− cells, monocytes, progenitor cells, or a cell line.

Another aspect of the disclosure provides a population of cells produced by a method according to the preceding paragraphs. Another aspect of the disclosure provides a pharmaceutical composition including cells produced by a method according to the preceding paragraphs.

Another aspect of the disclosure provides a method of manufacturing a cell therapy product comprising one or more transduced cells. The method includes (i) providing a system for transducing cells comprising a hollow fiber defining an intra-capillary space extending from a first end to a second end, (ii) loading a population of cells and a viral or non-viral vector into the intra-capillary space, resulting in transduction of one or more cells in the intra-capillary space, (iii) harvesting a population of cells comprising one or more transduced cells from the intra-capillary space.

This aspect of the disclosure may include one or more of the following optional features. In some implementations, the population of cells are selected from αβ T cells, γδ T cells, NK cells, HSCs, macrophages, dendritic cells and iPSCs. In some configurations, the viral or non-viral vector comprises a recombinant receptor. In some configurations, the recombinant receptor is a chimeric antigen receptor (CAR).

In some examples, the transduced cells comprise a recombinant receptor on the surface of cells. In some implementations, the chimeric antigen receptor includes an extracellular ligand-binding domain that targets a tumor antigen selected from one or more of CD44, CD19, CD20, CD22, CD23, CD30, CD89, CD123, CS-1, ROR1, mesothelin, c-Met, PSMA, Her2, GD-2, CEA, MAGE A3 TCR, EGFR, HER2/ERBB2/neu, EPCAM, EphA2, CEA and BCMA.

In some configurations, the method includes a step of isolating the transduced cells. In some configurations, the method further includes a step of expanding the harvested cells in a bioreactor. In some implementations, the method further includes a step of cryopreserving the harvested cells in a suitable cryopreservation medium. In some implementations, the system includes a first port in fluid communication with the intra-capillary space adjacent to the first end and a second port in fluid communication with the intra-capillary space adjacent to the second end.

In some examples, loading the viral or non-viral vector into the intra-capillary space includes loading the viral or non-viral vector from at least one of the first end and the second end of the intra-capillary space. In some implementations, loading the viral or non-viral vector into the intra-capillary space includes loading the viral or non-viral vector from each of the first end and the second end of the intra-capillary space. In some configurations, loading the cells into the intra-capillary space includes loading the cells from at least one of the first end and the second end of the intra-capillary space. In some implementations, loading the cells into the intra-capillary space includes loading the cells from each of the first end and the second end of the intra-capillary space.

Various aspects of the disclosure are described in detail in the following sections. The use of sections is not meant to limit the disclosure. Each section can apply to any aspect of the disclosure. In this application, the use of “or” means “and/or” unless stated otherwise. As used herein, the singular forms “a”, “an”, and “the” include both singular and plural referents unless the context clearly dictates otherwise.

### BRIEF DESCRIPTION OF DRAWINGS

FIG. 1A illustrates a hollow fiber system including a hollow fiber according to the present disclosure.

FIG. 1B illustrate a horizontal cross-section of the hollow fiber taken along Line 1B-1B of FIG. 1A, where the hollow fiber is loaded with cells and viral or non-viral vector.

FIG. 1C illustrates the vertical cross-section of the hollow fiber taken along Line 1C-1C of FIG. 1A, where the hollow fiber is loaded with cells and viral or non-viral vector.

FIG. 1D is schematic view of an example of a hollow fiber filter module including a plurality of hollow fibers according to the present disclosure.

FIG. 2A illustrates a hollow fiber system including a hollow fiber showing fluid flow direction during cells and viral vector loading.

FIG. 2B illustrates a horizontal cross-section of the hollow fiber showing fluid flow direction during cells and viral or non-viral vector loading.

FIG. 2C illustrates a vertical cross-section of the hollow fiber showing fluid flow direction during cells and viral of non-viral vector loading.

FIG. 3A illustrates a hollow fiber system including a hollow fiber showing fluid flow direction during introduction of viral or non-viral vector into target or host cells.

FIG. 3B illustrates a horizontal cross-section of the hollow fiber showing fluid flow direction during introduction of viral or non-viral vector into target or host cells.

FIG. 3C illustrates a vertical cross-section of the hollow fiber showing fluid flow direction during introduction of viral or non-viral vector into target or host cells.

FIG. 4A illustrates a hollow fiber system including a hollow fiber showing fluid flow direction during harvesting of cells.

FIG. 4B illustrates a horizontal cross-section of the hollow fiber with cells and viruses showing fluid flow direction during harvesting of cells.

FIG. 4C illustrates a vertical cross-section of the hollow fiber showing fluid flow direction during harvesting of cells.

FIG. 5 illustrates retrovirus transduced T cells under different transduction conditions.

FIG. 6 illustrates viability of T cells following their transductions under different conditions.

FIG. 7 illustrates retrovirus transduced NK cells under different transduction conditions.

FIG. 8 illustrates lentivirus transduced T cells under different transduction conditions.

FIG. 9 illustrates a technical layout of a semi-automated hollow fiber system for cell therapy transduction.

### DETAILED DESCRIPTION

### Current State of the Art

Transduction is a process through which viruses infect the cells of a target or host cell. Viruses naturally undergo the transduction process and have evolved to be very efficient at introducing genetic material into target cells. In order for transduction to occur, virus particles must come in physical contact with their target cells to first bind, enter, and finally introduce genetic material into the target cells. Binding occurs through specific protein-protein interactions, with the correct proteins needed on both the virus and target cell.

Cell therapies take advantage of the natural transduction process, using virus particles modified for safety and functionality as a delivery vehicle (vector) for introducing therapeutic genes into a patients cells. Viral vector transduction is currently the most frequently used method in cell therapy manufacturing for introducing therapeutic genetic material into a cell.

Current industry approaches to viral transduction include static transduction systems, the use of chemical enhancers, and spinoculation. Each of these current industry approaches is further described below.

Viral transduction under static conditions is the most prevalent manner in which viral transductions are currently performed. Under standard static transduction methods, most transductions are performed in standard culture flasks or bags under static culture conditions. In this manner, viral vector is suspended in media that can be about 100-1000s-fold deeper than the diameter of a single cell. Transduction using standard static methods face various problems that result in inefficient transduction of the cells. For example, using static methods results in the presence of small vector particles that remain in suspension and are unable to reach target cells. This occurs, at least in part, because large cells quickly sediment to the floor of culture vessels. The end result using the static culture methods for transduction is that only a small fraction of vector particles are capable of reaching cells through diffusion alone. As a result, transduction efficiency is low and the quantity of viral vector needs to be high to achieve appreciable cellular transduction. This is because viral vector binding to a target cell is determined by receptor/ligand expression and physical contact. The transduction rate is thus proportional to the local concentration of virus for a given cell. Requiring large quantities of viral vector to achieve a satisfactory transduction rate can be costly and also create inefficiencies in the overall cell therapy manufacturing process.

Another standard method for cellular transduction involves the use of chemical enhancers that in turn increase the binding rate of the vector to the cell. The use of methods that rely on chemical enhancers however is also expensive and removal of the chemical enhancer creates an added barrier in the manufacturing process.

Another standard method for cellular transduction is the use of spinoculation. Spinoculation refers to centrifugal inoculation of cells. Spinoculation reduces the volume occupied by cells. This technique has been shown to have various negative aspects including, for example, damage to cells, difficulty in scaling up, and it is generally less effective for small vectors.

Yet another method for enhancing the transduction efficiency of viruses, particularly retroviruses, is by use of a cell adhesive substance that binds to retroviruses, such as fibronectin or a fibronectin fragment CH-296 [RETRONECTIN(registered) (recombinant human fibronectin fragment) or retronectin]. This method requires adding a solution containing a retroviral vector to a vessel coated with retronectin followed by incubation for a certain period of time to allow only the viral vector to bind onto retronectin, removing supernatant, which contains inhibitory substances against virus infection, and then adding target cells. Coating of vessel surface with retronectin is tedious and makes this method rather costly. In addition, this method is difficult to scale up when gene transfer into a large amount of cells is required.

Cell Transduction Using Hollow Fiber System

The present disclosure relates to highly efficient methods of transducing cells using hollow fiber systems such as, for example, tangential fluid flow methods that allow for automated or semi-automated, high-efficiency cell transduction that can be applied to both lentivirus, retrovirus and other viral and non-viral vectors. The methods described herein provide an approach towards hollow fiber transduction that bypasses the limitations of current state-of-the-art methods.

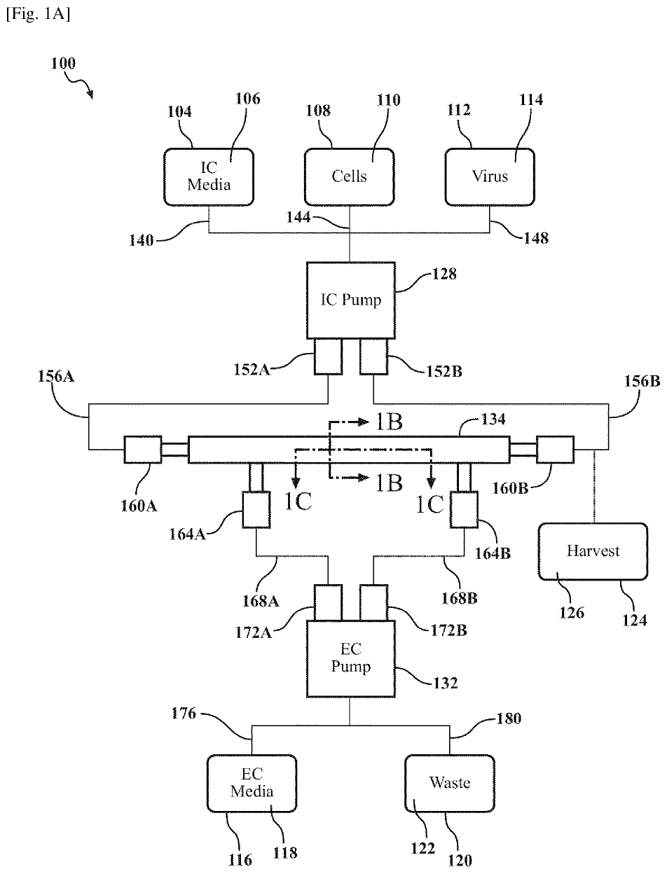

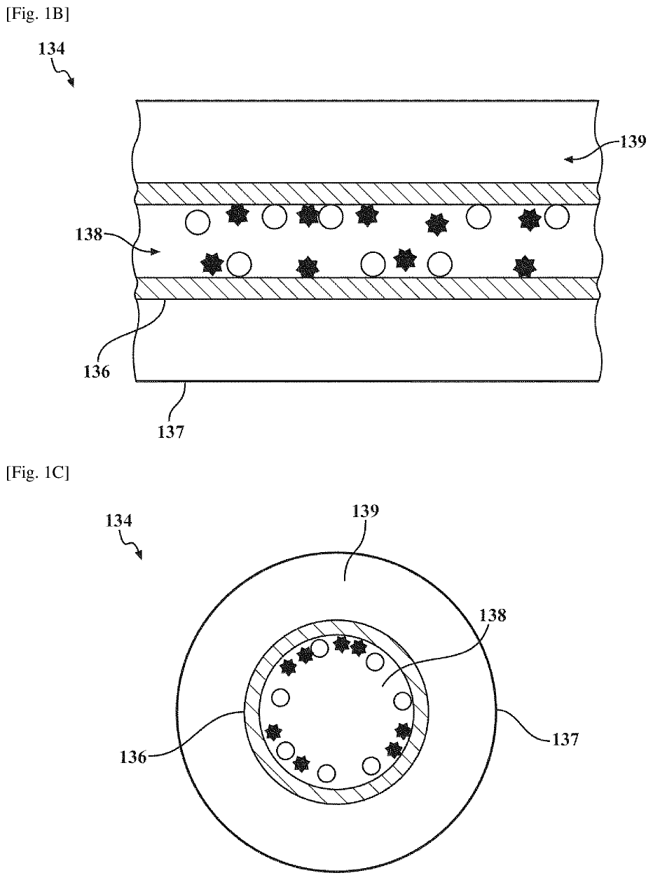

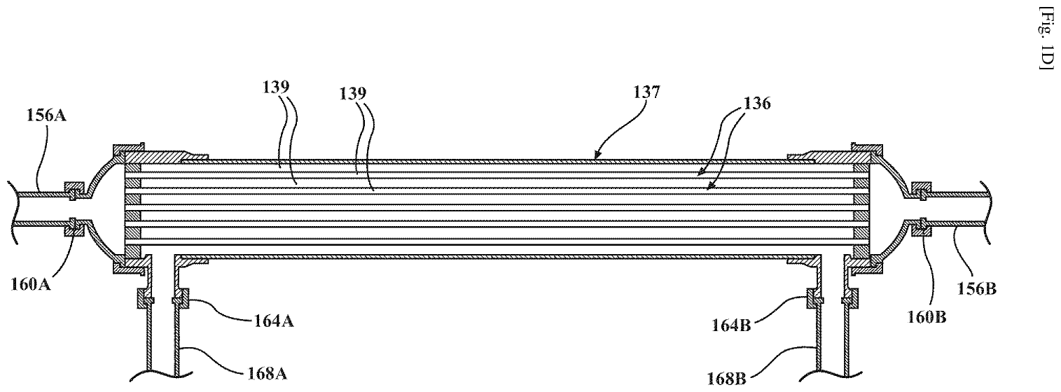

FIG. 1A illustrates a hollow fiber system 100 including one or more hollow fibers that are integrated within a custom designed pump/valve-based architecture. In some embodiments of the present disclosure, the hollow fiber system 100 includes an intra-capillary media container 104, a cell container 108, a virus container 112, an extra-capillary media container 116, a waste container 120, a harvest container 124, an intra-capillary pump 128, an extra-capillary pump 132, and a filter module 134 including one or more hollow fibers 136. As described in greater detail below, the filter module 134 provides a convenient means for introducing various materials into the hollow fiber system 100 and retrovirus materials.

The intra-capillary media container 104 contains intra-capillary media or transduction media 106, and is connected to the intra-capillary pump 128 through a transduction media conduit 140. The cell container 108 contains cells 110 and is connected to the intra-capillary pump 128 through a cell conduit 144. The cells may include B-cells, T cells, NK (natural killer) cells, monocytes, other lymphoid cells or progenitor cells.

The virus container 112 contains viruses or vector particles 114, and is connected to the intra-capillary pump 128 through a virus conduit 148. The vector 114 may include viral particles. In other examples, the viruses may include nucleic acid vectors. In some examples, the viruses are derived from a lentivirus, a retrovirus, an adenovirus, an adeno-associated virus, or a hybrid virus. In some embodiments, the viruses may include retroviruses or lentiviruses. In some examples, non-viral vector or vectors are used instead of viruses. Here, the non-viral vectors may include liposomes, lipid particles, carbon, non-reactive metals, gelatin, polyamine nanospheres, and/or inorganic nanoparticles. Additional examples of non-viral vectors include, for example spheroplasts, red blood cell ghosts, colloidal metals, inorganic nanoparticles, DEAE Dextran plasmids, among others, or a combination thereof. In some embodiments, inorganic nanoparticles are calcium phosphate nanoparticles.

While the present disclosure shows all three containers 104, 108, 112 independently connected to the intra-capillary pump 128 by way of the conduits 140, 144, 148, respectively, two or more of the containers 104, 108, 112 may share a common conduit. For example, all three containers 104, 108, 112 may connected to the intra-capillary pump 128 through a single conduit. In another embodiment, the cell container 108 and the virus container 112 may be connected to the intra-capillary pump 128 through a common conduit independent of the transduction media conduit 140.

The intra-capillary pump 128 receives one or more of the intra-capillary media 106, the cells 110, and the vector particles 114, and supplies them at a desired rate to the filter module 134. In the example shown, the intra-capillary pump 128 includes a first outlet 152A and a second outlet 152B in fluid communication with the filter module 134. The first outlet 152A is fluidly coupled to with the filter module 134 through a first intra-capillary conduit 156A and the second outlet 152B is fluidly coupled to the filter module 134 through a second intra-capillary conduit 156B. As shown, the filter module 134 is connected to the first intra-capillary conduit 156A through a first intra-capillary port 160A disposed at a first end of the filter module 134 and to the second intra-capillary conduit 156B through a second intra-capillary port 160B disposed at an opposite, second end of the filter module 134. The intra-capillary ports 160A and 160B may include valves operable to selectively regulate the passage of fluid/media into the filter module 134.

With continued reference to FIG. 1A, the extra-capillary media container 116 contains an extra-capillary or harvest media 118 and the waste container 120 is configured to receive fluid waste 122 from the filter module 134. The extra-capillary pump 132 is configured to provide fluid flow between the filter module 134 and each of the extra-capillary media container 116 and the waste container 120. Here, the extra-capillary pump 132 is connected with the extra-capillary media container 116 through an extra-capillary media conduit 176 and is connected with the waste container 120 through a waste conduit 180. The extra-capillary pump 132 includes two or more pump ports 172A, 172B that are each connected to the filter module 134 via a respective extra-capillary port 164A, 164B, which may include valves configured to regulate the flow of the extra-capillary media 118 and the waste 122 into and out of the filter module 134. A first extra-capillary port 164A connects the filter module 134 to a first extra-capillary pump port 172A of the extra-capillary pump 132 through a first extra-capillary conduit 168A. A second extra-capillary port 164B connects the filter module 134 to a second extra-capillary pump port 172B of the extra-capillary pump 132 through a second extra-capillary conduit 168B.

Each of the intra-capillary pump 128 and the extra-capillary pump 132 may include any type of pump operable to provide a fluid flow between the various containers 104, 108, 112, 116, 120 and the filter module 134. While the illustrated example shows each pump 128, 132 embodied as a single pump, other embodiments of the system 100 may include a plurality of intra-capillary pumps 128 and/or a plurality of extra-capillary pumps 132 each operable to provide fluid to or from one or more of the containers 104, 108, 112, 116, 120. The pumps 128, 132 may be embodied as manual pumps, such as syringes, or as powered pumps, such as metering pumps. Optionally, flow from each of the containers 104, 108, 112, 116, 120 to each of the pumps 128, 132 may be regulated by one or more valves implemented in the conduits 140, 144, 148, 176, 180 or containers 104, 108, 112, 116, 120. In other examples, each conduit 140, 144, 148, 176, 180 may be discretely connected to an independent pump 128, 132, whereby flow from each container 104, 108, 112, 116, 120 is directly regulated by the operation of the respective pump 128, 132.

FIG. 1B illustrates a horizontal cross-section of a simplified example of a hollow fiber 136. The horizontal cross-section is a cross-section of the hollow fiber 136 taken along Line 1B-1B as shown in FIG. 1A. The hollow fiber 136 may be enclosed within a casing 137 and form an example of the filter module 134. As shown, the space within the hollow fiber 136 defines an intra-capillary space 138 and the space outside of the hollow fiber 136 defines an extra-capillary space 139. For example, the extra-capillary space 139 is the space between the hollow fiber 136 and the casing 137. While the illustrated example shows a single hollow fiber 136 defining the intra-capillary space 138, it will be appreciated that there may be a plurality of the hollow fibers 136 arranged in parallel and cooperatively defining the intra-capillary space 138, such as in the example shown in FIG. 1D. One example of a filter module 134 is a MicroKros Hollow Fiber from Repligen, or the like. With continued reference to FIG. 1A, the first and second intra-capillary ports 160A, 160B are fluidly coupled the intra-capillary space 138 at opposite ends of the hollow fiber 136 while the first and second extra-capillary ports 164A, 164B are fluidly-coupled to the extra-capillary space 139 at opposite ends of the casing 137.

FIG. 1C illustrates a vertical cross-section of the hollow fiber 136 of the present disclosure. The vertical cross-section is a cross-section of the hollow fiber 136 taken along Line 1C-1C as shown in FIG. 1A. The vertical cross-section also illustrates the hollow fiber 136 disposed within the casing 137. The hollow fiber 136 includes a membrane with a plurality of pores defining a filter passage between the intra-capillary space 138 and the extra-capillary space 139. As set forth above and shown in FIG. 1D, a plurality of the hollow fibers 136 may be implemented in the filter module 134 where all of the hollow fibers 136 is contained within the casing 137. Here, each hollow fiber 136 defines a discrete portion of the intra-capillary space 138.

In one embodiment, the hollow fiber 136 is cylindrical and has a diameter of 500 μm. In some embodiments, the hollow fiber is cylindrical in shape. In some examples, the diameter of the hollow fiber is greater than about 80 μm, 100 μm, 150 μm, 200 μm. In some embodiments, the hollow fiber diameter is about 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, or about 1,000 μm.

In some embodiments, the hollow fiber 136 includes a membrane having a plurality of pore sizes. In one embodiment, the pore size of the membrane is 750 kD. In some examples, the pore size of the membrane of the hollow fiber 136 may be between about 50 and 100 kDa. In some examples, the pore size of the membrane of the hollow fiber 136 is greater than about 50 kDa. In some embodiments, the pore size of the membrane of the hollow fiber 136 is about 100 kDa to about 200 kDa. In some examples, the pore size of the membrane of the hollow fiber 136 is about 300 kDa, 400 kDa, 500 kDa, 30 nm, 40 nm, 50 nm, 100 nm, 200 nm, 300 nm, 400 nm, 500 nm, 600 nm, 700 nm, 800 nm, 900 nm, or 1 μm.

In some embodiments, the hollow fiber membrane includes polysulfone (PS), modified polyethersulfone (mPES), mixed cellulose ester (ME), polyethersulfone (PES), or mixtures thereof. In some examples, the hollow fiber membrane includes ceramic/s, metals or mixtures thereof. Optionally, the hollow fiber membrane does not include retronectin, fibronectin, and/or polybrene (i.e., is free of retronectin, fibronectin, and/or polybrene). In some embodiments, the introduction of the vector 114 into the cells 110 can be augmented by coating the membrane of the hollow fiber 136 with a compound. In some embodiments, the hollow fiber 136 is coated with retronectin. In some implementations, the hollow fiber 136 is coated with fibronectin. In some configurations, the membrane of the hollow fiber 136 is coated with polybrene. In some examples, the hollow fiber is coated with a mixture of retronectin, fibronectin, and/or polybrene.

Viral Transduction Process Using Hollow Fiber System

As explained in greater detail below, viral or non-viral vector introduction into a cell using the hollow fiber system 100 according to the present disclosure generally includes the following three steps: 1) loading cells and viral or non-viral vector into the intra-capillary space 138, 2) introduction of viral or non-viral vector into cells within the intra-capillary space 138, and 3) harvesting of cells and viral or non-viral vector from the intra-capillary space 138. Fluid flow direction can be adjusted at each step.

In some examples, harvested cells including transduced immune cells are transferred to a suitable bioreactor or culture vessel for expansion. Transduced cells are then expanded for 3-20 days in a suitable culture medium and then washed and suspended in final formulation buffer and cryopreserved in a suitable formulation for future therapeutic use.

In some examples, once harvested, transduced cells are separated from untransduced cells and vector by using suitable means in the art, e.g., affinity isolation of the transduced cells from vector and untransduced cells using an antibody that binds the chimeric antigen receptor (CAR) being expressed on the cells of the transduced cells or use of flow cytometry. Other suitable means in the art that may be used include, but are not limited to, size exclusion separation or some other method such as use of a column, membrane etc. to separate cells from vector. Once cells are isolated, separated or removed following the harvest step, cells may either be expanded and then cryopreserved or be cryopreserved following the harvest step and the cryopreserved cells may be subsequently used for future therapeutic use.

Fluid Flow Direction During Cells and Viral or Non-Viral Vector Loading

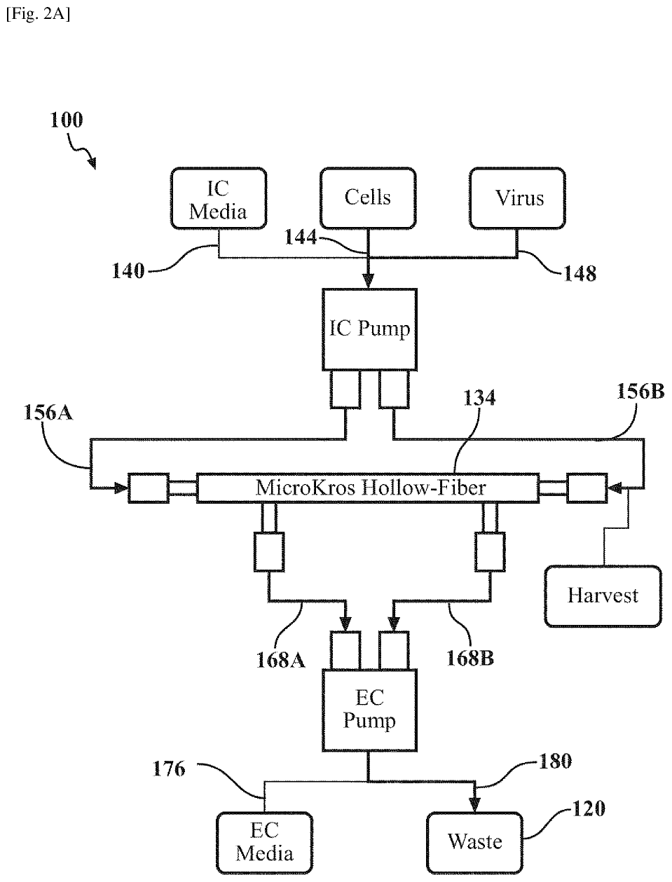

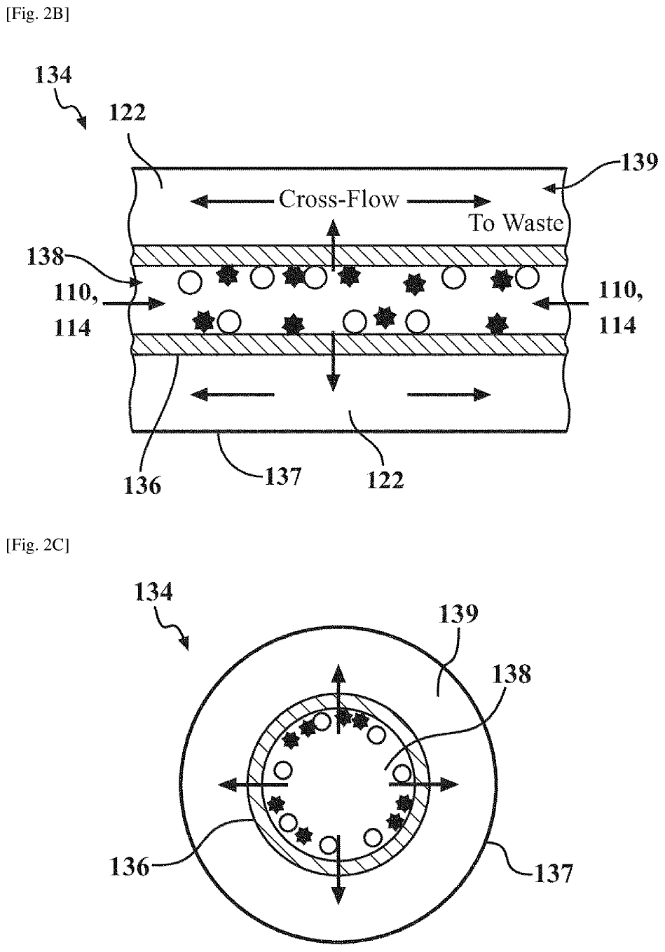

FIGS. 2A-2C illustrate the configuration and fluid flow direction of the hollow fiber system 100 during a cells and vector loading process. The direction of arrows within the conduits 140, 144, 148, 156A, 156B, 168A, 168B, 176, and 180; the intra-capillary space 138; the hollow fiber 136; and the extra-capillary space 139 indicate the fluid flow directions during the loading process. As shown in FIG. 2A, during the cell and vector loading process the intra-capillary pump 128 receives a flow of the cells 110 from the cell container 108 and a flow of the vector 114 from the virus container 112, but does not receive the intra-capillary media 106 from the intra-capillary media container 104. Thus while each of the containers 104, 108, 112 may be fluidly coupled to the intra-capillary pump 128, flow from each of the containers may be selectively controlled (e.g., turned on and off) via one or more valves.

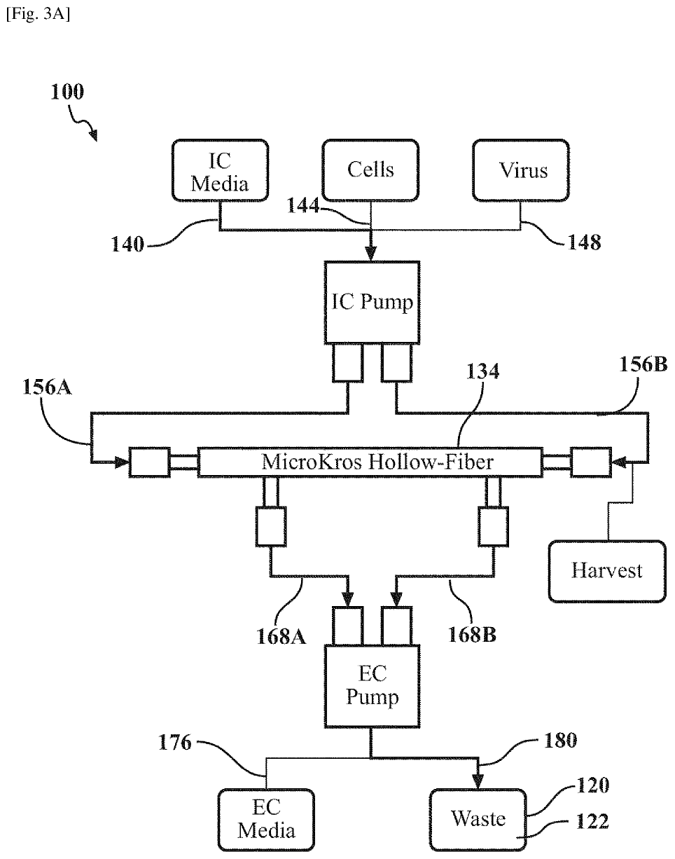

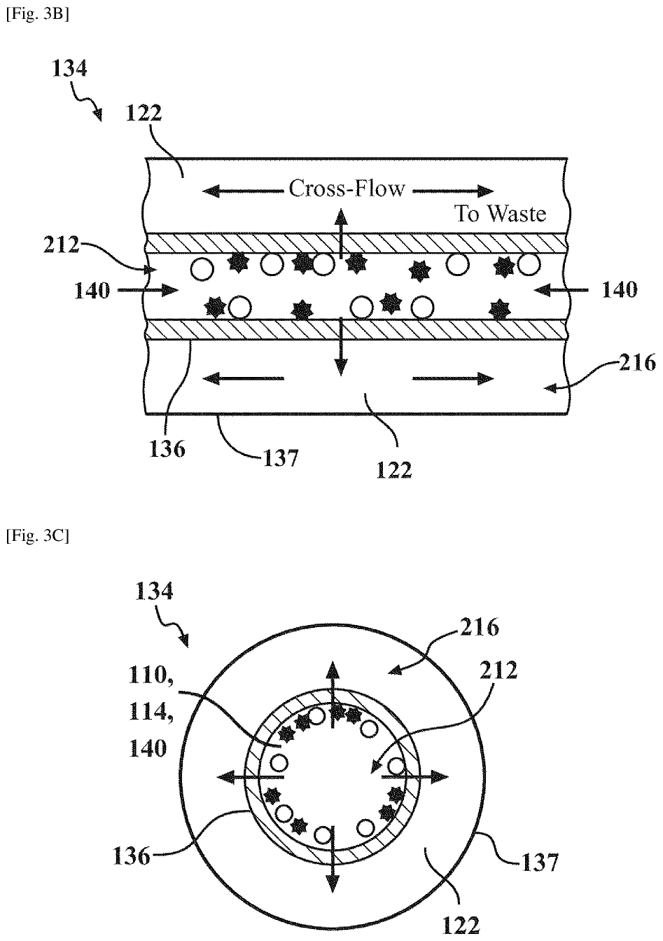

With continued reference to FIG. 2A, the intra-capillary pump 128 provides the cells 110 and the vector 114 to the intra-capillary space 138 via each of the first and second intra-capillary conduits 156A, 156B. As previously discussed, the intra-capillary conduits 156A, 156B may be connected to the intra-capillary space 138 via the intra-capillary ports 160A, 160B disposed at opposite ends of the hollow fiber filter module 134. Thus, cells 110 and vector 114 are introduced to the intra-capillary space 138 of the hollow fiber 136 from opposite ends of the hollow fiber 136 via the intra-capillary conduits 156A, 156B to create a counter-flow of the cells 110 and vector 114 within the intra-capillary space 138. As the cells 110 and vector 114 flow from opposite ends of the intra-capillary space 138, the counter-flows of the cells 110 and vector 114 collide and/or coalesce at a common region within the intra-capillary space 138 to define a transduction zone. Thus, during a transduction step, described below with respect to FIGS. 3A-3C, the cells 110 may be transduced within a localized region of the intra-capillary space 138 based on the counter-flow.

During the loading step, the cells 110 and the vector 114 may be provided to the intra-capillary space 138 simultaneously. In other examples, the cells 110 may be provided to the intra-capillary space 138 prior to providing the vector 114. Conversely, in some examples, the vector 114 may be provided to the intra-capillary space 138 before the cells 110. In another example, the cells 110 and vector 114 may be intermittently and alternatively provided to the intra-capillary space 138 through both ports 160A, 160B such that a layering of the cells 110 and vector 114 is provided within the intra-capillary space 138. Optionally, the cells 110 and the vector 114 may be loaded through one of the ports 160A, 160B while the other one of the ports 160A, 160B is in a closed state.

In some embodiments, cells are loaded into the hollow fiber at a concentration ranging between 1×10³ to 1×10¹⁰ cells/ml. In some embodiments, cells are loaded into the hollow fiber at a concentration of about 1×10⁶ to 1×10⁹ cells/ml. In some embodiments, the cells are loaded into the hollow fiber at a concentration of about 1×106, 1×10⁷ cells/ml, 2×10⁷ cells/ml, 3×10⁷ cells/ml, 4×10⁷ cells/ml, 5×10⁷ cells/ml, 6×10⁷ cells/ml, 7×10⁷ cells/ml, 8×10⁷ cells/ml, 9×10⁷ cells/ml or 1×10⁸ cells/ml.

In some embodiments, viral particles are loaded at a concentration ranging between 1×10⁶ IU virus/ml and 1×10⁹ IU virus/ml. In some embodiments, viruses are loaded at a concentration of about 1×10⁷ IU virus/ml, 2×10⁷ IU virus/ml, 3×10⁷ IU virus/ml, 4×10⁷ IU virus/ml, 5×10⁷ IU virus/ml, 6×10⁷ IU virus/ml, 7×10⁷ IU virus/ml, 8×10⁷ IU virus/ml, 9×10⁷ IU virus/ml, 1×10⁸ IU virus/ml, or 1×10⁹ IU virus/ml.

In some embodiments, the flow rate for loading viral or non-viral vector is a function of a size of an inner surface area of the membrane of the hollow fiber 136. In some examples, the flow rate per square centimeter of inner surface area of the membrane of the hollow fiber 136 ranges from 0.25 ml/min/cm² to 100 ml/min/cm². In some embodiments, the constant flow rate for loading cells into the hollow fiber is between 0.25 ml/min/cm² to 100 ml/min/cm². For example, in some implementations, the constant flow rate is about 0.25 ml/min/cm², 0.5 ml/min, 1 ml/min/cm², 5 ml/min/cm², 10 ml/min/cm², 15 ml/min/cm², 20 ml/min/cm², 25 ml/min/cm², 30 ml/min/cm², 35 ml/min/cm², 40 ml/min/cm², 45 ml/min/cm², 50 ml/min/cm², 55 ml/min/cm², 60 ml/min/cm², 65 ml/min/cm², 70 ml/min/cm², 75 ml/min/cm², 80 ml/min/cm², 85 ml/min/cm², 90 ml/min/cm², 95 ml/min/cm² or 100 ml/min/cm².

In some embodiments, the cells and viral particles are loaded into the hollow fiber intra-capillary space at a multiplicity of infection (MOI) of about 0.25, 0.5, 1.0, 1.5, 2.0, 2.5, 3.0, 3.5, or 4.0. Accordingly, in some embodiments, the cells and viral particles are loaded at an MOI of about 0.25. In some embodiments, the cells and the viral particles are loaded at an MOI of about 0.5. In some embodiments, the cells and the viral particles are loaded at an MOI of about 1.0. In some embodiments, the cells and the viral particles are loaded at an MOI of about 1.5. In some embodiments, the cells and the viral particles are loaded at an MOI of about 2.0. In some embodiments, the cells and the viral particles are loaded at an MOI of about 2.5. In some embodiments, the cells and the viral particles are loaded at an MOI of about 3.0. In some embodiments, the cells and the viral particles are loaded at an MOI of about 3.5. In some embodiments, the cells and the viral particles are loaded at an MOI of about 4.0

With the cells 110 and the vector 114 loaded within the intra-capillary space 138, the hollow fiber 136 retains and concentrates the cells 110 and vector 114 within the intra-capillary space 138 of the hollow fiber 136. As a result, cells 110 and vector 114 are concentrated in the intra-capillary space 138 (e.g., on an interior surface of the membrane of the hollow fiber 136. Waste or fluid 122 from the cells 110 and vector 114 passes through the pores of the hollow fiber 136 from the intra-capillary space 138 to the extra-capillary space 139. As shown in FIG. 2B, the waste 122 flows in opposite directions through the extra-capillary space 139 to the extra-capillary ports 164A, 164B disposed at opposite ends of the filter module 134. Here, the opposing flow of the waste 122 towards the extra-capillary ports 164A, 164B results in a cross-flow of the outgoing flow of waste 122 relative to the incoming flow of cells 110 and vector 114. From the extra-capillary ports 164A, 164B, the waste 122 travels to the extra-capillary pump ports 172A, 172B of the extra-capillary pump 132 via the extra-capillary conduits 168A, 168B, and is then discharged by the pump 132 to the waste container 120 via the waste conduit 180.

Fluid Flow Direction During Viral or Non-Viral Vector Introduction

Once the cells 110 and the vector 114 are loaded into the intra-capillary space 138, the hollow fiber system 100 is configured to introduce the intra-capillary media 106 to the intra-capillary space to encourage transduction. The intra-capillary fluid loading step promotes cell 110 and vector 114 interaction in the intra-capillary space 138 (for example, on the interior surface of the membrane of the hollow fiber 136), which results in binding of the vector 114 to the cells 110 and consequent entry of the vector particle 114 into the cell 110. FIGS. 3A-3C illustrate the configuration and fluid flow direction of the hollow fiber system 100 during the transduction process. The direction of the arrows indicates the fluid flow directions for respective materials 122, 140 during the transduction process. As shown in FIG. 3A, during the transduction process, the cell container 108 and the virus container 112 are not in fluid communication with the intra-capillary pump 128, while the intra-capillary media container 104 is in fluid-communication with the intra-capillary pump 128. Accordingly, the intra-capillary pump 128 receives a flow of the intra-capillary media 106, but does not receive the cells 110 or the vector 114.

With continued reference to FIG. 3A, the intra-capillary pump 128 provides the intra-capillary media to the intra-capillary space 138 via each of the first and second intra-capillary conduits 156A, 156B to initiate the vector introduction. Thus, like the cells 110 and vector 114, the intra-capillary media 106 may be loaded in the intra-capillary space 138 from opposite ends of the hollow fiber 136. In one embodiment, the transduction time is about 90 minutes.

The intra-capillary media 106 may be provided to the intra-capillary space 138 using a continuous and constant fluid flow at low flow rates to prevent virus diffusion away from cells. In some embodiments, the constant flow rate for introduction of viral or non-viral vectors into cells is between 10 μl/min to 5 ml/min. In some embodiments, the constant flow rate for transducing vectors into cells is between 10 μl/min to 5 ml/min. For example, in some embodiments, the constant flow rate is about 10 μl/min, 25 μl/min, 50 μl/min, 100 μl/min, 250 μl/min, 500 μl/min, 750 μl/min, 1 ml/min, 2 ml/min, 3 ml/min, 4 ml/min, or 5 ml/min.

In some embodiments, cells and viral or non-viral vectors are subjected to fluid flow for between about 5 minutes to about several days. In some embodiments, the cells and viruses are subjected to fluid flow for between 5 minutes to about 18 hours. In some embodiments, the cells and viruses are subjected to fluid flow for between 60 minutes to about 120 minutes. In some embodiments, the cells and viruses are subjected to fluid flow for about 90 minutes. In some embodiments, the cells are further cultured in a hollow fiber system after transduction for several weeks.

During the transduction process, fluid enters the intra-capillary space of the filter module 134 via the ports 160A, 160B, and passes through the pores of the hollow fiber 136 from the intra-capillary space 138 and flows out to the extra-capillary space 139. Waste or fluid 122 from the transduction process passes through the pores of the hollow fiber 136 from the intra-capillary space 138 to the extra-capillary space 139. As shown in FIG. 3B, the waste 122 flows in opposite directions through the extra-capillary space 139 to the extra-capillary ports 164A, 164B disposed at opposite ends of the filter module 134. Here, the opposing flow of the waste 122 towards the extra-capillary ports 164A, 164B results in a cross-flow of the outgoing flow of waste 122 relative to the incoming flow of intra-capillary media 140. From the extra-capillary ports 164A, 164B, the extra-capillary pump 132 receives the waste 122 via the extra-capillary conduits 168A, 168B and then discharges the waste 122 to the waste container 120 via the waste conduit 180.

Fluid Flow Direction During Harvesting of Cells and Viral or Non-Viral Vector

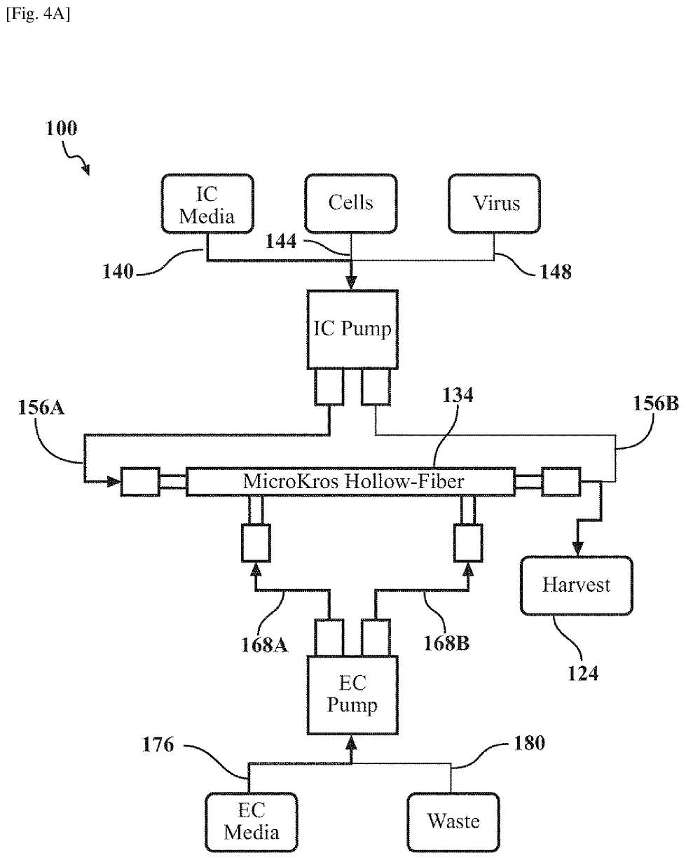

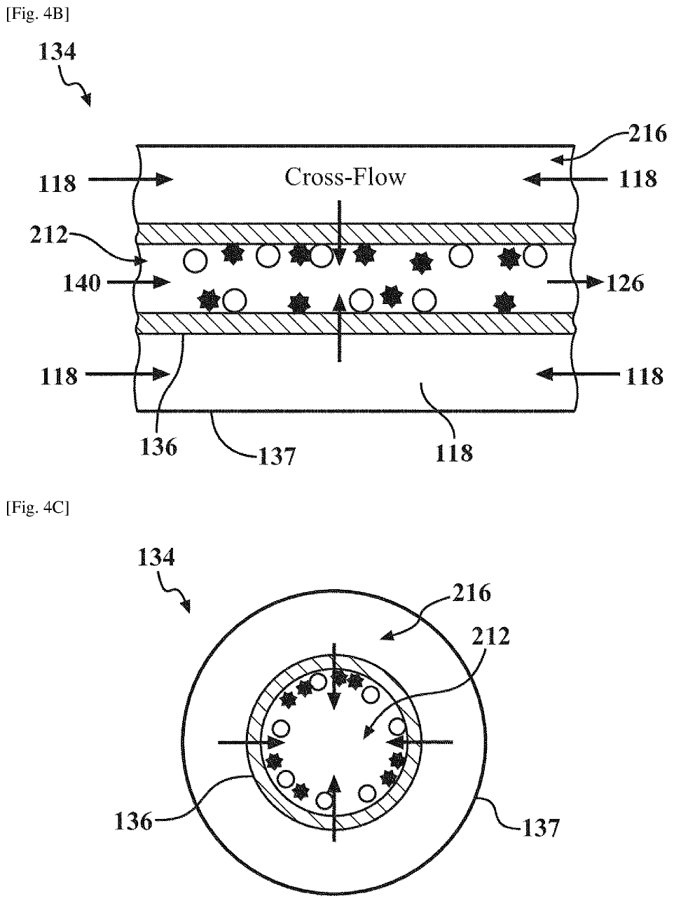

After the transduction process shown in FIGS. 3A-3C, the system 100 is configured to harvest the transduced cells 126 from the intra-capillary space 138. FIGS. 4A-4C illustrate a configuration and fluid flow direction for the hollow fiber system 100 during a cell harvesting process. The direction of the arrows indicates the fluid flow directions during the cell harvesting process. As shown in FIG. 4A, during the harvesting process the extra-capillary pump 132 provides a flow of the extra-capillary media 118 from the extra-capillary media container 116 to the extra-capillary space 139 via each of the extra-capillary ports 164A, 164B. As shown in FIGS. 4B and 4C, the extra-capillary media 118 passes from the extra-capillary space 139 to the intra-capillary space 138 to dislocate the transduced cells 126 from the intra-capillary space 138. For example, the extra-capillary media 118 is introduced to the extra-capillary space of the filter module 134 via each of the extra-capillary ports 164A, 164B to maximize dislocation of the transduced cells 126 from the interior surface of the membrane of the hollow fiber 136 into the intra-capillary space 138.

With continued reference to FIG. 4A, the intra-capillary pump 128 may also provide a flow of the intra-capillary media 106 (or other flushing fluid) from the intra-capillary media container 104 to the intra-capillary space 138 to flush the released transduced cells 126 from the intra-capillary space. However, unlike during the transduction process (FIGS. 3A-3C) where the intra-capillary media 106 is provided from both ends of the hollow fiber 136 via both of the intra-capillary ports 160A, 160B, during the harvesting process the intra-capillary media 106 is only provided via one of the intra-capillary ports 160A to initiate a unidirectional flow through the intra-capillary space 138. Repeated unidirectional fluid flow through the intra-capillary space enables harvesting of transduced cells 126 from the intra-capillary space 138 to the harvest container 124 via the other one of the intra-capillary ports 160B.

In some examples, the transduced cells 126 are harvested from the intra-capillary space 138 in a complete culture media and then transferred directly to a suitable bioreactor or culture vessel. The transduced cells are then expanded for an expansion period (e.g., 3-20 days) in a product-dependent culture buffer. Once expanded, the cells are washed and suspended within a final formulation buffer before being frozen for therapeutic use. In other examples, the transduced cells 126 may be harvested from the intra-capillary space 138 in the final formulation buffer. Here, the transduced cells 126 are introduced to a membrane, column, or other based process for size selection of target cells and removal of superfluous virus. The selected target cells are then frozen for future therapeutic use.

Retroviral and Lentiviral Transductions Using Semi-Automated Hollow Fiber System

### Example 1. Retronectin-Free Retroviral Transduction of T Cells Using Semi-Automated Hollow Fiber System

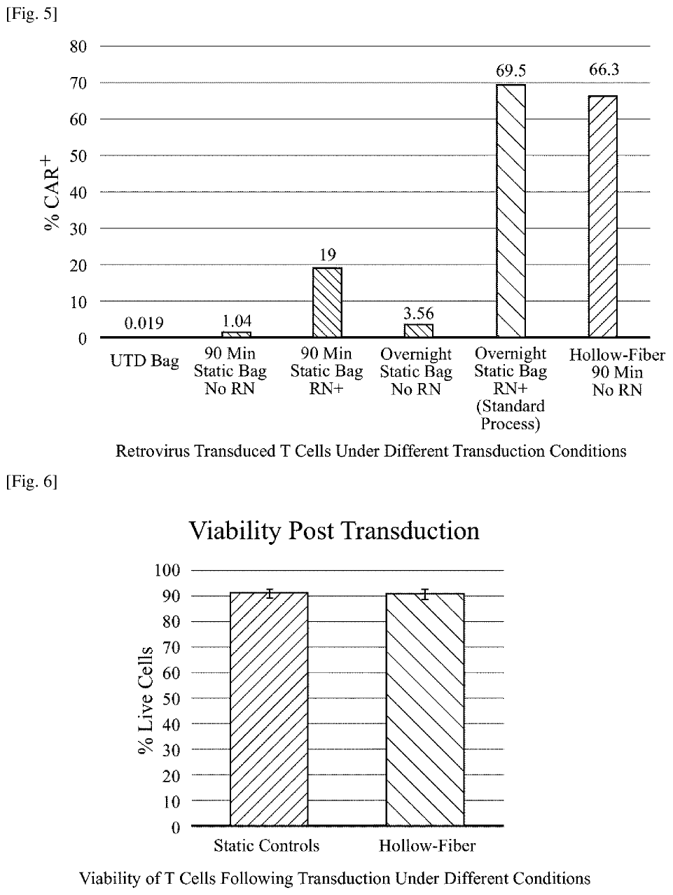

This example illustrates a study demonstrating retronectin-free retroviral transduction of T cells using a semi-automated hollow fiber system. This example compares the transduction rate achieved under six different conditions: a) an untransduced (UTD) static bag only, b) a static bag without retronectin (RN) coating with cells and virus co-incubated for 90 minutes, c) a static bag with retronectin coating incubated for 90 minutes, d) a static bag without retronectin coating with cells and virus co-incubated overnight, e) a static bag with retronectin coating with cells and virus co-incubated overnight (a standard process), and f) a semi-automated hollow fiber system with no retronectin with cells and virus co-incubated for 90 minutes. The comparative transduction rates for all six of conditions are illustrated in FIG. 5.

In this example, three-fold dilutions of the retrovirus were prepared to determine optimum infectious range. CD4/CD8 isolated T cells were thawed, and activated for 48 hours. In static control conditions, 7 million preactivated T cells at a 1 million cells/mL concentration were taken in a culture bag. These preactivated cells were then transduced either overnight or for 90 minutes with virus (MOI 2.5). Retronectin controls were prepared and cell bags were coated with retronectin overnight at 10 μg/mL. Retronectin-coated bags were preincubated with retroviruses for 2 hours.

In the semi-automated hollow fiber system 100, cells and viruses at MOI 2.5 were loaded into the filter module 134 and transduced for 90 minutes. No retronectin was used in the hollow fiber system 100. At the end of the 90-minute transduction process, cells and viruses were harvested from the filter module 134 followed by washing to remove viruses, and then the cells were plated in GREX-6M. Overnight transduced static bag cells also went through a similar process the following day. All cells were expanded for 5 days post-transduction and then harvested for flow analysis.

The data showed that retronectin-coated bags demonstrated higher transduction rates compared to the bags without retronectin coating when the cells were transduced for a similar interval of time. For instance, a static bag with retronectin coating that was incubated for 90 minutes showed a higher transduction rate compared to a static bag without retronectin coating and incubated for similar interval of time, as shown in FIG. 5. Similarly, a static bag with retronectin coating that was incubated overnight showed higher transduction rate compared to a static bag without retronectin coating incubated overnight, as shown in FIG. 5. The semi-automated hollow fiber system 100 without retronectin coating that incubated for 90 minutes demonstrated about the same transduction rate as that of the static bag with retronectin coating that incubated overnight (i.e., the standard process), as can be clearly appreciated in FIG. 5.

In addition, there was no appreciable difference in the viability of cells harvested from bags (i.e., static controls) and the cells harvested from the hollow fibers following transduction, as shown in FIG. 6. Similarly, there was no appreciable difference in the expansion or proliferation of cells between cells transduced in bags and cells transduced in hollow fiber system.

### Example 2. Retronectin-Free Retroviral Transduction of NK Cells Using Semi-Automated Hollow Fiber System

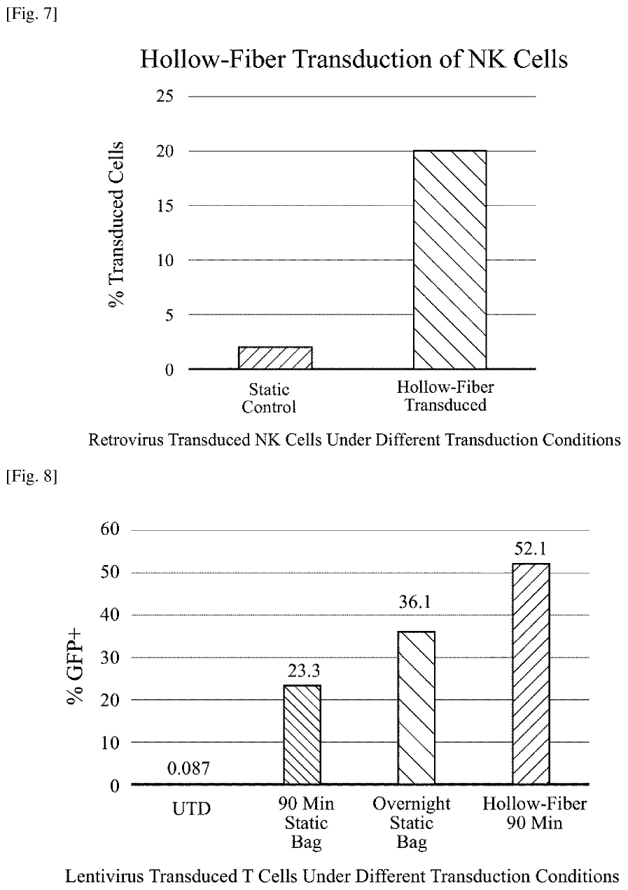

This example illustrates a proof-of-concept study demonstrating retroviral transduction of NK cells using a hollow fiber system. This example compares the transduction rates achieved under two different conditions: a) a static plate without retronectin coating, incubated for 90 minutes, b) a semi-automated hollow fiber system 100 without retronectin coating, incubated for 90 minutes. The comparative transduction rates for these two conditions are illustrated in FIG. 7.

In this example, retrovirus was prepared to the optimum infectious range. Fresh cord blood NK cells were isolated, and activated for 6 days before transduction. In static control condition, 5 million preactivated NK cells at a 1 million cells/mL concentration were transduced for 90 minutes with virus (MOI 2). In the semi-automated hollow fiber system, cells and viruses at MOI 2 were loaded into the hollow fiber and transduced for 90 minutes. At the end of the 90-minute transduction process, cells and viruses were harvested from the hollow fiber system 100 followed by washing to remove viruses, and then the cells were plated in tissue culture plates. Transduced static cells also went through a similar process. All cells were expanded for 9 days post-transduction, and then harvested for flow analysis.

The data showed that the hollow fiber system demonstrated higher transduction rates into NK cells compared to the static plate controls, as indicated in FIG. 7.

### Example 3. Lentiviral Transduction Using Semi-Automated Hollow Fiber System

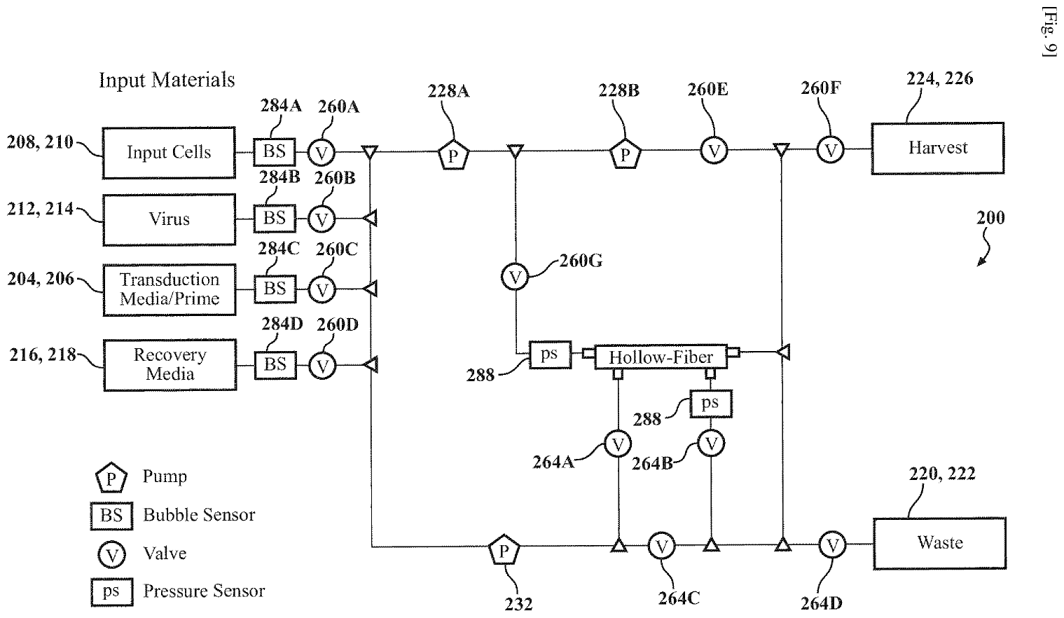

This example illustrates a proof-of-concept study demonstrating lentiviral transduction using a semi-automated hollow fiber system. This example compares the transduction rates achieved under four different conditions: a) an untransduced bag (i.e., static bag only), b) a static bag incubated for 90 minutes, c) a static bag incubated overnight, and d) a semi-automated hollow fiber incubated for 90 minutes. The comparative transduction rates for all these four conditions are illustrated in FIG. 8.

In this example, lentivirus vector with ZsGreen reporter was used. CD4/CD8 isolated T cells were thawed, and activated for 48 hours. A single vial of cells and virus mix [multiplicity of infection (MOI) of 1] was prepared and then aliquoted into separate vials to ensure equal MOI.

In static control conditions, 7 million preactivated T cells at 1 million cells/mL concentration were taken in a cell bag. These preactivated cells were then transduced either overnight or for 90 minutes with virus at MOI 1.

In the semi-automated hollow fiber system, the cells/virus mix was loaded into the hollow fiber and transduced for 90 minutes. At the end of the 90 minutes transduction process, cells and viruses were harvested from the hollow fiber followed by washing to remove viruses, and then the cells were plated in GREX-6M. Overnight transduced cells also went through a similar process the following day. All cells were expanded for 5 days post-transduction, and then harvested for flow analysis.

The static bag transduced overnight showed higher transduction rate compared to a static bag transduced for 90 minutes, as shown in FIG. 8. The semi-automated hollow fiber system 100 incubated for only 90 minutes demonstrated about 1.4-fold higher transduction compared to that of the static bag incubated overnight, as can be clearly seen in FIG. 8. In addition, there was no appreciable difference in viability of cells harvested from bags and the cells harvested from the hollow fiber following transduction. Similarly, there was no appreciable difference in the expansion between bags and hollow fiber following transduction.

Semi-Automated Hollow Fiber System for Cell Therapy Transduction

FIG. 9 illustrates a schematic layout of another example of a hollow fiber system 200 for cell therapy transduction. The layout features input materials 206, 210, 214, 218, output materials 222, 226, and a hollow fiber 236.

The input materials includes transduction media 206, cells 210, vector 214, and recovery/harvest media 218. Each container 204, 208, 212, 216 for the input materials is also connected to a bubble sensor 284 and a valve 260A-260D, that controls the flow of the input material 206, 210, 214 to the hollow fiber 236. The bubble sensors 284A-D detect the presence of bubbles in the input materials 206, 210, 214, 218 and help ensure that the hollow fiber 236 receives bubble-free input materials 206, 210, 214, 218.

The output materials include harvested cells 226 and waste 222. Each output material's container 220, 224 is also connected to one or more ports 164A, 164B that controls the flow of fluids/media to the output material containers 220, 224 from the hollow fiber 236.

The hollow fiber 236 is connected to several pumps 228A, 228B, 232 via valves 260E-260G, 264A-264D and pressure sensors 288. These pumps 228A, 228B, 232 control the rate of fluid flow to the intra-capillary space and extra-capillary space of the hollow fiber 236 during cells and viruses loading process, the transduction process, and harvesting process, which are carried out with the hollow fiber 236 in a similar manner as described above with respect to the hollow fiber 236.

The systems and methods disclosed herein markedly increase the efficiency of introduction of viral or non-viral vector into cells by increasing contact between the vector and target cells using a hollow fiber system. In this manner, large quantities of cells are exposed to sufficient vector concentrations that allow efficient transduction of the cells. This results in reduced time for transducing cells while also minimizing vector waste. Therefore, the disclosure provides systems and methods that not only reduce the total amounts of the vector used to achieve high transduction of the cells, but also reduces the transduction time significantly. Accordingly, in one aspect, the systems and methods described herein achieve efficient cellular transduction at a reduced cost compared to conventional transduction systems. Additional benefits of the systems and methods disclosed herein include an increased amount of transduced cells, less virus consumed during the transduction process, reduced process time, and reduced manufacturing costs. This in turn benefits patients at least because the methods allow for faster processing time, and the creation of a more potent therapeutic.

The methods described herein use a hollow fiber system that enables tangential fluid flow from one side of the hollow fiber to the other side of the hollow fiber. The hollow fiber system comprises one or more hollow fibers. The hollow fiber comprises a porous cylindrical surface that allows the tangential fluid flow across the membrane. The tangential fluidic flow brings the vector in contact/proximity of cells that contributes to increased viral transduction efficiency.

The porous cylindrical surface of the hollow fiber enables flow-through of fluid and small molecules, but at the same time does not allow flow through of cells and larger molecules. Thus, in some embodiments described herein, the hollow fiber comprises pore sizes that selectively allow certain molecules to flow through and out of the hollow fiber, while at the same time retaining cells and other larger molecules. Furthermore, the hollow fiber as described herein can be tailored to have a porosity between 50 kDa to 1 μm to further augment the desired flow characteristics to achieve high efficiency of introduction of viral or non-viral vector into the target or host cells. The porosity of the hollow fiber can also be tailored based on the size of the viral or non-viral vector to be used. In some embodiments, the pore size of the hollow fiber is one-fourth of the size of the viral or non-viral particles. In some embodiments, the pore size of the hollow fiber is one-third of the size of the viral or non-viral particles. In some embodiments, the pore size of the hollow fiber is half of the size of the viral or non-viral particles. An additional parameter of the hollow fiber than can be adjusted to further optimize the efficiency of introduction of viral or non-viral vectors into target or host cells is the diameter of the hollow fiber itself.

Uses of Transduced Cells

The cells introduced with viral or non-viral vectors using the methods described herein allows for using the cells for any purpose that a modified cell can have. The modified cells retain high viability (e.g., greater than 70%, 75%, 80%, 85%, or 90%, or up to 98%) and can be used for a variety of applications, such as for cell therapy purposes such as, for example, in adoptive cell therapy applications.

In some embodiments, the viability and proliferation the transduced cells using hollow fiber system is same as that of the transduced cells using an overnight static condition.

Adoptive Cell Therapy

The methods described herein can be used, among other things, to genetically engineer cells for use in various therapeutic methods, including for example for use in adoptive cell therapy applications.

Adoptive cell therapy (“ACT”) refers to an infusion into patients of autologous or allogeneic cells to treat disease. Various cell types can be used for ACT-based therapies, such as B-cells, T cells, NK− cells, monocytes, progenitor cells, or cell lines. The progenitor cells can be isolated directly from a patient or from a non-patient donor. The progenitor cells include, for example, adult stem cells and pluripotent cells such iPSCs derived from a patient or non-patient donor. In some embodiments, ACT uses genetically modified hematopoietic stem cell (“HSC”) transplantation.

Hematopoietic stem cell (“HSC”) transplantation, one category of ACT methods, involves the infusion of autologous or allogeneic stem cells to reestablish hematopoietic function in patients whose bone marrow or immune system is damaged or defective. It also allows the introduction of genetically modified HSCs, for example, to treat congenital genetic diseases. In typical HSC transplantation, the HSCs are obtained from the bone marrow, peripheral blood or umbilical cord blood.

In some embodiments, cells obtained from the peripheral blood are genetically engineered for use in ACT methods. Peripheral blood is used for autologous transplantations because of high stem cell and progenitor cell content as compared to bone marrow or cord blood. Moreover, HSCs obtained from peripheral blood show faster engraftment following transplantation. Because HSCs in the peripheral blood are present at low concentrations, the donor is typically treated with a mobilizing agent, such as granulocyte colony stimulating factor (G-CSF) or granulocyte macrophage colony stimulating factor (GM-CSF), which affects adhesion of HSCs to the bone marrow environment and releases them into the peripheral blood.

In some embodiments, the methods described herein are used to genetically modify T cells for T cell immunotherapy-based ACT methods. T cell immunotherapy is another category of ACT method and involves the infusion of autologous or allogeneic T lymphocytes that are selected and/or engineered ex vivo to target specific antigens, such as for example tumor-associated antigens. The T lymphocytes are typically obtained from the peripheral blood of the donor by leukapheresis. In some T cell immunotherapy methods, the T lymphocytes obtained from the donor, such as tumor infiltrating lymphocytes (“TIL”s), are expanded in culture and selected for antigen specificity without altering their native specificity. In other T cell immunotherapy methods, T lymphocytes obtained from the donor are engineered ex vivo, typically by transduction with viral expression vectors, to express chimeric antigen receptors (“CAR”s) of predetermined specificity. CARs typically include an extracellular domain, such as the binding domain from a scFv, that confers specificity for a desired antigen; a transmembrane domain; and one or more intracellular domains that trigger T-cell effector functions, such as the intracellular domain from CD3δ or FcRγ, and, optionally, one or more co-stimulatory domains drawn, e.g., from CD28 and/or 4-1BB. In still other T cell immunotherapy methods, T lymphocytes obtained from the donor are engineered ex vivo, typically by transduction with viral expression vectors, to express T cell receptors (“TCR”s) that confer desired specificity for antigen presented in the context of specific HLA alleles.

In some embodiments, the methods described herein are used to genetically modify hematopoietic stem cell (HSCs). In some embodiments, the HSCs are subject to additional treatments to expand the population of HSCs or manipulated by recombinant methods described herein to introduce heterologous genes or additional functionality to the allogeneic HSCs prior to transplantation into the recipient subject. In certain embodiments, the additional treatment leads to maturation of the HSCs.

HSCs obtained from a donor, either autologous or allogeneic, can be subject to additional treatments prior to transplantation into a recipient subject. In some embodiments, the HSCs are treated to expand the population of HSCs, for example by culturing one or more HSCs in a suitable medium.

In some embodiments, the HSCs, either autologous or allogeneic, are manipulated by recombinant methods to introduce heterologous genes by the methods disclosed herein. Such genetic manipulations can be used to correct genetic defects, and/or introduce additional functionality to the HSCs prior to transplantation. In some embodiments, a functioning wild type gene is introduced into the HSC to correct a genetic defect, for example, congenital hematopoietic disorders (e.g., β-thalassemia, Fanconi anemia, hemophilia, sickle cell anemia, etc.); primary immunodeficiencies (e.g., adenosine deaminase deficiency, X-linked severe combined immunodeficiency, chronic granulomatous disease, Wiskott-Aldrich syndrome, Janus kinase 3 deficiency, purine nucleoside phosphorylase (PNP) deficiency, leukocyte adhesion deficiency type 1, etc.); and congenital metabolic diseases (e.g., mucopolysaccharidosis (MPS) types I, II, III, VII, Gaucher disease, X-linked adrenoleukodystrophy, etc.). In certain embodiments, the HSCs are subjected to gene manipulation by recombinase systems, such as genome editing using CRISPR/Cas9 system or Cre/Lox recombinases. For example, the recombinase systems can be used to ablate genes or correct gene defects. In various embodiments, other methods of altering the functionality of HSCs include, among others, introduction of antisense nucleic acids, ribozymes, and RNAi.

In some embodiments, progenitor cells or cell lines are modified by introducing viral or non-viral vectors into the progenitor cell or cell lines. Any suitable progenitor or cell line can be used in accordance with the methods and systems described herein. As non-limiting examples, suitable progenitor cells include, for example, cells isolated directly from a patient or from a non-patient donor. The progenitor cells include, for example, adult stem cells and pluripotent cells such iPSCs derived from a patient or non-patient donor. Various cell lines can also be used with the methods and systems described herein, and include, for example, mammalian cell lines, of human or non-human origin.

Other features, objects, and advantages of the present disclosure are apparent in the examples that follow. It should be understood, however, that the examples, while indicating embodiments of the present disclosure, are given by way of illustration only, not limitation. Various changes and modifications within the scope of the disclosure will become apparent to those skilled in the art from the examples.

### Definitions

Adoptive Cell Therapy: As used herein, the term “adoptive cell therapy,” “adoptive cell transfer” or “ACT” refers to the transfer of cells into a patient in need thereof. The cells can be derived and propagated from the patient in need or could have been obtained from a non-patient donor. In some embodiments, the cell is an immune cell, such as a lymphocyte. Various cell types can be used for ACT such as, for example, a T-cells, CD8+ cells, CD4+ cells, NK-cells, delta-gamma T-cells, regulatory T-cells and peripheral blood mononuclear cells. In some embodiments, the cells are genetically modified to introduce a chimeric antigen receptor (CAR).

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a stated value as well as a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Chimeric Antigen Receptor (CAR): As used herein, the term “chimeric antigen receptor” or “CAR” engineered receptors which can confer an antigen specificity onto cells transduced using methods described herein (for example immune cells such as NK cells, iPSC derived NK cells (iNK cells), T cells such as naive T cells, central memory T cells, effector memory T cells, gamma delta T cells, T regulatory cells or combinations thereof). CARs are also known as artificial T-cell receptors, chimeric T-cell receptors or chimeric immunoreceptors. In various embodiments, a CAR described herein may include one or more of an antigen-specific targeting domain, an extracellular domain, a transmembrane domain, optionally one or more co-stimulatory domains, and an intracellular signaling domain.

Cryopreservation: As used herein, the term “cryopreservation” generally refers to a freezing a biological material (e.g., a population of cells or transduced cells) to low enough temperatures, such that chemical processes, which might otherwise damage the material are halted thereby preserving the material. Cryopreserved cells maintain viability for an extended period of time in the frozen state, such as for 1, 5, 10 or more years in the cryopreserved state. The cryopreserved cells, once thawed, are able to propagate both for in vitro and in vivo applications.

Host cell or Target Cell: As used herein, the terms “host cell” or “target cell” includes cells that are not transfected, not infected and not transduced. In some embodiments, the terms “host cell” or “target cell” includes transfected, infected, or transduced with a recombinant vector or a polynucleotide of the disclosure. Host cells may include packaging cells, producer cells, and cells infected with viral vectors. In particular embodiments, host cells infected with viral vector of the disclosure are suitable for administering to a subject in need of therapy. In some embodiments, the target cell is a stem cell or progenitor cell. In certain embodiments, the target cell is a somatic cell, e.g., adult stem cell, progenitor cell, or differentiated cell. In preferred embodiments, the target cell is a hematopoietic cell, e.g., a hematopoietic stem or progenitor cell. In some embodiments, the target cell includes B-cells, T cells, NK-cells, monocytes or progenitor cells. In some embodiments, the target cell is a mammalian cell, an insect cell, bacterial cell, or fungal cell.

Mammalian Cell Lines

In some embodiments, a “host cell” or “target cell” includes a cell line. Various cell lines are known in the art and are suitable for use with the disclosure. Suitable cell lines include, for example, mammalian cell lines, of human or non-human origin.

Any mammalian cell or cell type susceptible to cell culture, and to expression of polypeptides, may be utilized in accordance with the present disclosure as a host cell or target cell. Non-limiting examples of mammalian cells that may be used in accordance with the present disclosure include human embryonic kidney 293 cells (HEK293), HeLa cells; BALB/c mouse myeloma line (NSO/1, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells+/−DHFR (CHO, Urlaub and Chasin, Proc. Natl. Acad. Sci. USA, 77:4139 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W136, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); MRC 5 cells; FS4 cells; and a human hepatoma line (Hep G2). In some embodiments, a suitable mammalian cell is not a endosomal acidification-deficient cell.

Additionally, any number of commercially and non-commercially available hybridoma cell lines that express polypeptides or proteins may be utilized in accordance with the present disclosure. One skilled in the art will appreciate that hybridoma cell lines might have different nutrition requirements and/or might require different culture conditions for optimal growth and polypeptide or protein expression, and will be able to modify conditions as needed.

Non-Mammalian Cell Lines

Any non-mammalian derived cell or cell type susceptible to cell culture, and to expression of polypeptides, may be utilized in accordance with the present disclosure as a host cell. Non-limiting examples of non-mammalian host cells and cell lines that may be used in accordance with the present disclosure include cells and cell lines derived from Pichia pastoris, Pichia methanolica, Pichia angusta, Schizosacccharomyces pombe, Saccharomyces cerevisiae, and Yarrowia lipolytica for yeast; Sodoptera frugiperda, Trichoplusis ni, Drosophila melangoster and Manduca sexta for insects; and Escherichia coli, Salmonella typhimurium, Bacillus subtilis, Bacillus lichenifonnis, Bacteroides fragilis, Clostridia perfringens, Clostridia difficile for bacteria; and Xenopus Laevis from amphibian.

Functional equivalent or derivative: As used herein, the term “functional equivalent” or “functional derivative” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cellbased systems, the term may be used to refer to events that occur within a living cell (as opposed to, for example, in vitro systems).

Non-viral vectors: As used herein, the term “non-viral vectors” includes for example, nanoparticles, liposomes, lipid particles, carbon, non-reactive metals, gelatin and/or polyamine nanospheres.

Primary Cell: The term, “primary cell,” refers to cells that are directly isolated from a subject and which are subsequently propagated.

Polypeptide: The term, “polypeptide,” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of the disease, disorder, and/or condition.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Vector: As used herein, the term “vector” means the combination of any carrier and any foreign gene(s). The vector may include non-viral vectors, viral vectors, among others, and any combination thereof. For example, non-viral vectors may include but are not limited to liposomes, spheroplasts, red blood cell ghosts, colloidal metals, calcium phosphate, DEAE Dextran plasmids, among others, or a combination thereof. The viral vectors may include but are not limited to retroviral vectors, lentiviral vectors, pseudotype vectors, adenoviral vectors, adeno-associated viral vectors, hybrid virus, among others, and any combination thereof.

Transduction: As used herein, the term “transduction” means a process whereby foreign DNA is introduced into another cell via a viral vector. Various viral vectors are known in the art and include, for example, retroviral vectors, lentiviral vectors, pseudotype vectors, adenoviral vectors, adeno-associated viral vectors, among others, and any combination thereof.

Transfection: As used herein, the term “transfection” means a process of introducing nucleic acids into cells by non-viral methods. In some embodiments, the methods described herein are suitable for transfection of a cell of interest.

The recitation of numerical ranges by endpoints herein includes all numbers and fractions subsumed within that range (e.g. 1 to 5 includes 1, 1.5, 2, 2.75, 3, 3.9, 4 and 5). It is also to be understood that all numbers and fractions thereof are presumed to be modified by the term “about.”

Various aspects of the disclosure are described in detail in the following sections. The use of sections is not meant to limit the disclosure. Each section can apply to any aspect of the disclosure. In this application, the use of “or” means “and/or” unless stated otherwise. As used herein, the singular forms “a”, “an”, and “the” include both singular and plural referents unless the context clearly dictates otherwise.

1. A system for introducing a vector into cells, the system comprising:
a filter module defining an intra-capillary space and an extra-capillary space separated from the intra-capillary space by a porous membrane;
a pair of intra-capillary ports fluidly coupled to opposite ends of the intra-capillary space and each receiving a transduction media, cells, and a vector; and
a pair of extra-capillary ports coupled to opposite ends of the extra-capillary space and in fluid-communication with a source of extra-capillary media and a waste container.

2. The system of claim 1, further comprising a harvest container in fluid communication with at least one of the intra-capillary ports.

3. The system of claim 1, further comprising an intra-capillary pump operable to provide a flow of each of the transduction media, the cells, and the vector to at least one of the intra-capillary ports.

4. The system of claim 3, wherein the intra-capillary pump is operable in a first state to provide the cells and the vector to the intra-capillary ports during a first period of time and in a second state to provide the transduction media to the intra-capillary ports during a second period of time.

5. The system of claim 1, further comprising a waste container in communication with the extra-capillary space through at least one of the extra-capillary ports.

6. The system of claim 1, further comprising an extra-capillary pump operable to provide a flow of the extra-capillary media to each of the extra-capillary ports.

7. The system of claim 1, further comprising an extra-capillary pump operable to provide a flow of a waste fluid from the extra-capillary ports to the waste container.

8. The system of claim 1, wherein the porous membrane is cylindrical.

9. The system of claim 1, wherein the porous membrane comprises pores that allow particles with a size of less than about 50 kDa to pass through the pores from the intra-capillary space.

10. The system of claim 1, wherein the intra-capillary space defines a transduction zone.

11. A system for introducing a viral or a non-viral vector into cells, the system comprising:
a hollow fiber defining an intra-capillary space extending from a first end to a second end; and
a casing enclosing the one or more hollow fibers to define an extra-capillary space between the hollow fiber and the casing from the first end to the second end, the casing comprising a first port in fluid communication with the intra-capillary space adjacent to the first end and a second port in fluid communication with the intra-capillary space adjacent to the second end;
a transduction media source in fluid communication with the intra-capillary space through each of the first port and the second port;
a cell source including cells and in fluid communication with the intra-capillary space through each of the first port and the second port; and
a virus source including a viral or non-viral vector and in fluid communication with the intra-capillary space through each of the first port and the second port.

12. The system of claim 11, further comprising a harvest container in fluid communication with the intra-capillary space through at least one of the first port and the second port.

13. The system of claim 11, further comprising an intra-capillary pump including an inlet in fluid communication with each of the transduction media source, the cell source, and the virus source.

14. The system of claim 13, wherein the intra-capillary pump includes a first outlet in fluid communication with the intra-capillary space through the first port and a second outlet in fluid communication with the intra-capillary space through the second port.

15. The system of claim 11, wherein the casing includes a third port in communication with the extra-capillary space and the system further comprises:
a waste container in communication with the extra-capillary space through the third port.

16. The system of claim 15, further comprising an extra-capillary media source in fluid communication with the extra-capillary space through the third port.

17. The system of claim 16, wherein the third port is disposed adjacent to the first end of the intra-capillary space and the system further comprises a fourth port in fluid communication with the extra-capillary space and disposed adjacent to the second end of the intra-capillary space.

18. The system of claim 17, wherein each of the waste container and the extra-capillary media source are in communication with the extra-capillary space through each of the third port and the fourth port.

19. The system of claim 11, wherein the hollow fiber comprises a plurality of hollow fibers.

20. The system of claim 11, wherein the hollow fiber comprises pores that allow particles with a size of less than about 50 kDa to pass through the pores from the intra-capillary space.

21-62. (canceled)
